(19) United States
(12) Patent Application Publication
(10) Pub. No.: US 2025/0000873 A1
(43) Pub. Date: Jan. 2, 2025

(54) INHALED ILOPROST FOR RESCUE TREATMENT AND TREATMENT AS NEEDED IN PULMONARY HYPERTENSION

(71) Applicant: JUSTUS-LIEBIG-UNIVERSITAET GIESSEN, Giessen (DE)

(72) Inventors: Werner Seeger, Biebertal (DE); Tobias Gessler, Wettenberg (DE)

(21) Appl. No.: 18/690,532

(22) PCT Filed: Sep. 1, 2021
(86) PCT No.: PCT/EP2021/074903

§ 371 Date: Mar. 8, 2024

(51) Int. Cl. A61K 31/5578; A61K 9/00; A61P 9/12

(52) U.S. Cl. CPC A61K 31/5578; A61K 9/0078; A61P 9/12

(57) ABSTRACT

Provided herein are methods for treating pulmonary hypertension. The methods include administering to a subject an effective amount of iloprost on demand or as rescue medication (also referred to as pro re nata, PRN), wherein iloprost is administered to the subject via inhalation using a portable soft mist inhaler. In preferred embodiments, the soft mist inhaler is the Respimat™ or the Medspray™ wet aerosol inhaler.

A method of treating a patient suffering from pulmonary hypertension, comprising:
(a) providing a portable and pre-filled soft mist inhaler adapted for delivering an effective amount of iloprost; and
(b) administering to the patient the effective amount of iloprost by inhalation on an as-needed basis.

### FIELD OF INVENTION

The present invention relates to methods and compositions for the treatment of patients by Iloprost aerosol therapy.

### BACKGROUND OF THE INVENTION

Iloprost is a synthetic prostacyclin analogue accounting for the biological activity of prostacyclin (also referred to as PGI2 or epoprostenol) which is known to dilate blood vessels, inhibit fibroblast growth, reduce platelet aggregation, and to possess anti-inflammatory and anti-mitogenic properties. According to IUPAC the chemical name of Iloprost is 5-{(E)-(1S,5S,6R,7R)-7-hydroxy-6[(E)-(3S,4RS)-3-hydroxy-4-methyl-1-octen-6-inyl]-bicyclo[3.3.0]octan-3-ylidene}pentanoic acid. Iloprost is approved for the therapy of pulmonary (arterial) hypertension, scleroderma, Raynaud's phenomenon and certain types of ischemia. Iloprost is available as aqueous solution in glass ampoules with different concentrations (10 μg/ml to 100 μg/ml), additionally containing trometamol, ethanol 96%, sodium chloride, hydrochloric acid (for pH adjustment) and water for injections. For the treatment of pulmonary hypertension, Iloprost is marketed as Ventavis™ in two concentrations of 10 μg/ml (Ventavis-10) and 20 μg/ml (Ventavis-20).

Pulmonary hypertension (PH) is a severe and potentially life-threatening disease defined by an increase in mean pulmonary arterial pressure above 25 mmHg.

Common signs and symptoms of pulmonary hypertension include shortness of breath (dyspnea), exercise intolerance, fatigue, dizziness or syncope, chest pressure or pain, edema formation, cyanosis, tachycardia and heart palpitations. Pulmonary hypertension is currently classified by the WHO into the following five groups: Group 1, Pulmonary arterial hypertension (PAH); Group 2, Pulmonary hypertension due to left heart disease; Group 3, Pulmonary hypertension due to lung disease and/or hypoxia; Group 4, Pulmonary hypertension due to pulmonary artery obstructions; Group 5, Pulmonary hypertension with unclear and/or multifactorial mechanisms. Deciphering the pathophysiological background of PH has facilitated the development of specific PH medication over the last decades, especially for group 1 and 4 PH. Currently, there are several PH-specific medications available, addressing the three principal signaling pathways of pulmonary vasoregulation: the prostacyclin-pathway with epoprostenol (intravenous), Iloprost (inhaled, intravenous), treprostinil (inhaled, intravenous, subcutaneous, oral), beraprost (oral) and selexipag (oral); the nitric oxide-pathway with sildenafil (oral), tadalafil (oral), vardenafil (oral) and riociguat (oral); the endothelin-pathway with bosentan (oral), ambrisentan (oral) and macitentan (oral). These mainly vasodilatory drugs have considerably improved therapy of P(A)H including amelioration of clinical symptoms, deceleration of disease progression and prolonged survival. Despite this progress, however, there is still no cure for this disease. Patients often report to suffer from the handicapping symptoms, even when treated by one or more PH-specific maintenance therapies. PH continues to severely impair patient's quality of life and often hampers their participation in social and occupational life. Everyday activities are cumbersome, with day-to-day and hour-to-hour differences in exercise capacity; some patients experience pulmonary hypertensive crises when exercising or when drug action levels off. These symptoms are often accompanied by anxieties and mental-health problems avoiding being alone or avoiding physical exertion.

Inhaled Iloprost is approved in many countries for aerosol therapy of pulmonary (arterial) hypertension as monotherapy or in addition to pre-existing P(A)H-specific medication, e.g. bosentan, to improve exercise capacity and symptoms. Inhaled iloprost (Ventavis™, Bayer Vital GmbH, Actelion Pharmaceutical, Janssen) is marketed in two strengths (Ventavis-10, Ventavis-20), and administered from 6 to 9 times per day using the Breelib™ nebulizer, the I-Neb™ AAD™ inhaler system or the Venta-Neb™ nebulizer. The target dose of Ventavis™ treatment is 2.5 μg or 5 μg iloprost as delivered at the mouthpiece of the nebulizer, subject to patient's tolerability. The 2.5 μg or 5 g dose per inhalation session should be administered 6 to 9 times per day according to the individual need and tolerability. Breelib™ is a handheld, battery-powered, breath activated, vibrating mesh inhalation system. When filling Ventavis-10 (1 ml ampoule) or Ventavis-20 (1 ml ampoule) into the medication chamber of the device, a dose of 2.5 μg or 5 μg Iloprost is delivered at the mouthpiece, respectively. The duration of an inhalation session with the Breelib™ nebulizer is approximately 3 minutes, according to the breathing pattern of the inhaling patient. The I-Neb™ AAD™ system is a portable, hand-held, vibrating mesh technology nebulizer monitoring the breathing pattern to determine the aerosol pulse time required to deliver the pre-set dose of 2.5 μg or 5 μg. This device can be used for the administration of Ventavis-10 or Ventavis-20 (each in 1 ml ampoule), the delivered dose being controlled by the medication chamber in combination with a control disc. Ventavis-10 is routinely used to deliver 2.5 μg or 5.0 μg Iloprost at the mouthpiece of the nebulzser within 3.2 or 6.5 minutes, respectively. Only patients who are maintained at the 5 μg dose and who have repeatedly experienced extended inhalation times with Ventavis-10 may be considered suitable for switching to Ventavis-20. Venta-Neb™ is a portable ultrasonic battery-powered nebulizer guiding the inhaling patient by an optical and an acoustic signal. For each inhalation session with the Venta-Neb™, the content of one 2 ml ampoule of Ventavis-10 is transferred into the nebulizer medication chamber immediately before use. Two programs can be operated, with program 1 delivering 5 μg Iloprost at the mouthpiece within 25 inhalation cycles and program 2 2.5 μg within 10 inhalation cycles.

Subject to the European Public Assessment Report (EPAR) product information, the posology and method of administration of Ventavis™ includes dosing of inhaled Iloprost according to the individual need and tolerability. Therefore, the use of inhaled Iloprost on an as-needed basis (also referred to as pro re nata (PRN), meaning as circumstances arise or dictate) is foreseen and designated by the drug label, contrary to the claims of Weers et al. in U.S. Pat. No. 10,912,778 B2 (“Methods for treatment of pulmonary hypertension”). With the available inhalation systems for Ventavis™ (Breelib™, I-Neb™ AAD™ and Venta-Neb™) a PRN use of Ventavis™ is theoretically possible, although cumbersome. A PRN dosed medication for PH has several requirements. Firstly, pharmacodynamics and pharmacokinetics of the PRN drug must allow rapid onset of drug action after administration (within few minutes), and must provide pulmonary selectivity avoiding potential side effects in the systemic circulation. Secondly, the method and way of drug administration must be convenient and easy to use, portable and safe. In the case of inhaled Iloprost, its suitable pharmacodynamics and pharmacokinetics for PRN use have extensively been documented, e.g. in Gessler et al. (Pulm Circ. 2017, 7 (2): 505-513, “The safety and pharmacokinetics of rapid Iloprost aerosol delivery via the BREELIB nebulizer in pulmonary arterial hypertension) and Olschewski et al. (Chest 2003; 124(4): 1294-1304, “Pharmacodynamics and pharmacokinetics of inhaled Iloprost, aerosolized by three different devices, in severe pulmonary hypertension”). The three recommended nebulizers for the aerosol administration of Ventavis™, although portable, do not facilitate the PRN use of the drug. For each treatment session, there are many different steps required for preparation and performance of the inhalation: entrainment of the non-pocket-sized devices and the separate Ventavis™ ampoules, preparation of the devices, opening of a single glass ampoule of Ventavis™, transfer of the drug into the medication chamber of the devices by a pipette or syringe, inhalation time of three minutes or more, removal of the residual drug and cleaning of the devices.

Patent EP 000002701683 B1 “Administration of Iloprost as aerosol bolus” provided the basis for the development of the Breelib™ nebulizer for Ventavis™ aerosol therapy. The patent focuses on vibrating mesh nebulizers capable of delivering iloprost within a period of two minutes or less. The use of soft mist inhalers is mentioned in a general manner, however, no detailed description of methods to use such soft mist inhalers for Iloprost inhalation therapy are disclosed. U.S. Pat. No. 10,912,778 B2 “Methods for treatment of pulmonary hypertension” claims “a method of treating pulmonary hypertension, the method comprising administering to a subject in need thereof an effective amount of a vasodilator, wherein the vasodilator is administered via inhalation pro re nata using a portable inhale” focusing on inhaled formulations of phosphodiesterase-5 inhibitors. Iloprost as possible vasodilator as well as different soft mist inhalers as possible devices are mentioned in the patent, however, no detailed descriptions of methods to use Iloprost along with such soft mist inhalers for Iloprost PRN therapy are disclosed. Furthermore, the pro re nata use of Iloprost is already foreseen and disclosed by prior art and prior publications (see for example European Public Assessment Report (EPAR) product information, available at https://www.ema.europa.eu/en/medicines/human/EPAR/ventavis.

Until now, there is no specific PRN therapy available in pulmonary hypertension, contrary to therapeutic options in asthma or COPD. For example, reliever medication is an essential category of asthma therapy, provided to all asthmatic patients for as-needed relief of breakthrough symptoms, including during worsening asthma or exacerbations, or for short-term prevention of exercise-induced asthma. A special type of PRN therapy in asthma is the maintenance and reliever regimen, also called ‘MART’ or ‘SMART’ therapy, with patients receiving inhaled corticosteroid-formoterol as their regular twice-daily or once-daily maintenance treatment, and additionally taking doses by means of the same inhaler for relief of symptoms.

It is an object of the present invention to provide methods and compositions for administering inhalable Iloprost as PRN therapy which overcome at least one of the disadvantages and shortcomings known from conventional Iloprost aerosol therapy.

### SUMMARY OF THE INVENTION

Provided herein are methods and compositions to treat pulmonary hypertension by inhalation of an affective amount of Iloprost on an as-needed basis, also referred to as pro re nata, by portable, pre-filled soft mist inhalers. In preferred embodiments, the soft mist inhaler is the Respimat™ or the Medspray™ wet aerosol inhaler. Inhaled Iloprost is approved as Ventavis™ in many countries to treat pulmonary (arterial) hypertension. This prostacyclin analogue is administered on a regular basis 6 to 9 times per day by the use of different nebulizers. The recommended nebulizers do not facilitate the PRN use of inhaled Iloprost, their handling is cumbersome.

The inventions provides PRN inhaled Iloprost for the acute treatment of PH on an as-needed basis in therapy-naÏve patients or in patients treated by one or more PH-specific drugs on regular basis, to facilitate improvements in exercise tolerance and activities of daily living, to reduce symptoms of the disease, or to overcome acute pulmonary hypertensive crises. In preferred embodiments, PRN Iloprost is administered by the portable and pre-filled soft mist inhalers Respimat™ or Medspray™ allowing patients to inhale at anytime and anywhere an effective dose of Iloprost up to 5 μg.

Further aspects and embodiments will become clear on the basis of the detailed description below, the examples, and the patent claims.

### BRIEF DESCRIPTION OF THE DRAWINGS

FIG. 1 shows the mean pulmonary arterial pressure (PAP) in patients with pulmonary arterial hypertension after inhalation of 2.5 mg or 5 μg Iloprost (0 min: baseline before inhalation); n=4; mean±SEM; *p<0.05, Mann-Whitney rank-sum test.

FIG. 2 shows the pulmonary vascular resistance (PVR) in patients with pulmonary arterial hypertension after inhalation of 2.5 mg or 5 μg Iloprost (0 min: baseline before inhalation); n=4, mean±SEM; *p<0.05, Mann-Whitney rank-sum test.

FIG. 3 shows the mean systemic arterial pressure (SAP) in patients with pulmonary arterial hypertension inhalation of 2.5 mg or 5 μg Iloprost (0 min: baseline before inhalation); n=4, mean±SEM; ns: not significant, Mann-Whitney rank-sum test.

FIG. 4 shows the systemic vascular resistance (SVR) in patients with pulmonary arterial hypertension after inhalation of 2.5 mg or 5 μg Iloprost (0 min: baseline before inhalation); n=4; mean±SEM; ns: not significant, Mann-Whitney rank-sum test.

### DETAILED DESCRIPTION OF THE INVENTION

The invention provides methods and compositions for administering Iloprost as pro re nata (PRN, medication on demand or rescue medication, meaning as circumstances arise or dictate) therapy of pulmonary hypertension by pre-filled, portable and user-friendly soft mist inhalers.

Iloprost is also known by its chemical name 5-{(E)-(1S,5S,6R,7R)-7-hydroxy-6[(E)-(3S,4RS)-3-hydroxy-4-methyl-1-octen-6-inyl]-bicyclo[3.3.0]octan-3-ylidene}pentanoic acid, according to IUPAC. Iloprost solution is marketed as Ventavis™ for aerosol therapy of pulmonary hypertension. Ventavis™ solution contains iloprost together with trometamol, ethanol 96%, sodium chloride, hydrochloric acid (for pH adjustment) and water for injections in 1 or 2 ml glass ampoules. Currently, there are two strengths of Ventavis™ available, either containing 10 μg/ml iloprost (Ventavis-10) or 20 μg/ml iloprost (Ventavis-20). Additionally, in some countries an aqueous iloprost formulation (Ilomedin™) with an iloprost concentration of 100 μg/ml is available for infusion to treat several diseases of peripheral arteries, also containing trometamol, ethanol 96% (v/v), sodium chloride, hydrochloric acid (1 N) and water for injections as excipients.

Ventavis™ is approved in many countries for aerosol therapy of pulmonary (arterial) hypertension (P(A)H) as monotherapy or in addition to pre-existing P(A)H-specific medication, e.g. bosentan, to improve exercise capacity and symptoms. Ventavis™ solution is aerosolized and delivered to the inhaling patient by different nebulizers. According to the latest product information, the nebulizers Breelib™, I-Neb™ AAD™ inhaler system or the Venta-Neb™ are recommended as suitable devices for use of Ventavis™ by inhalation. These devices are typical nebulizers requiring multiple steps to perform the inhalation maneuver: opening of a glass ampoule containing 1 or 2 ml of Ventavis™, assembly of the nebulizer, transfer of Ventavis™ solution into the nebulization chamber of the nebulizer by a pipette or syringe, inhalation with a duration of at least three minutes for delivery of a nominal iloprost dose at the mouthpiece of 2.5 or 5.0 μg, removal of the residual solution in the nebulization chamber, cleaning of the different pieces of the nebulizer. In principle, the recommended nebulizers can be used independent of mains supply. A PRN use, as foreseen in the European Public Assessment Report (EPAR) product information, is theoretically possible, although cumbersome.

The use of soft mist inhalers for iloprost aerosol therapy has been suggested several times in the past. There are, however, no data or specific methods provided for the use of such devices in iloprost aerosol therapy.

A key feature of the present invention is to select soft mist inhalers (SMI) for administering iloprost as pro re nata therapy of pulmonary hypertension. In a preferred embodiment, the soft mist inhaler is the Respimat™ (Boehringer Ingelheim, Germany), in a further preferred embodiment the soft mist inhaler is the Medspray™ wet aerosol inhaler (Medspray, The Netherlands).

The Respimat™ soft mist inhaler is a hand-held, pocket-sized device generating a single-breath, inhalable aerosol with slow velocity and long spray duration. By forcing non-pressurized drug solution through a two-channel nozzle (uniblock) using mechanical power, the solution is accelerated and split into two converging jets which collide at a certain angle, causing the drug solution to disintegrate into respirable droplets. The mechanical energy for the aerosolization process is provided by rotating the bottom of the device by 180° building up tension in a spring around the flexible drug container. When actuated by the patient, energy from the spring is released and imposes pressure on the flexible container holding the liquid drug formulation, whereby a metered-dose of liquid is forced through two nozzles and dispersed into an inhalable aerosol. The Respimat™ is already marketed, and for example available for the aerosol administration of tiotropium in COPD.

The Medspray™ wet aerosol inhaler is a hand-held, preservative-free, non-pressurized metered dose device containing micro-engineered nozzles produced by wafer stepper lithography and etching techniques. The aerosol is produced according to the principle of Rayleigh break-up, with liquid being dispersed into droplets by pressing the drug solution through an array of nozzles with mechanical means. The drug solution can be stored in a container with a mechanical pump system or in pre-filled glass syringes with the soft mist nozzles already mounted. Different nozzles can be used to target a specific site in the respiratory tract. The mechanical energy for the aerosolization process is for example provided by a spring which is loaded and released by the patient.

For use in PRN iloprost aerosol therapy of pulmonary hypertension, the drug containers of the Respimat™ or Medspray™ soft mist inhalers are pre-filled with Ventavis-10 or Ventavis-20, or Ilomedin™ 100 μg/ml, or Ilomedin™ 100 μg/ml diluted with physiological saline, resulting in iloprost drug concentrations in the range of 10 μg/ml to 100 μg/ml.

The following is a non-exhaustive list of examples describing in detail and explaining the use of soft mist inhalers for unique application of inhaled iloprost as a PRN therapy for patients with PAH and other forms of PH.

### Example 1 Respimat™ and Ventavis-20

In an in vitro nebulization study, the feasibility of delivering iloprost solution by the Respimat™ was evaluated. The physical aerosol characteristics and the output of placebo Respimat™ and Respimat™ filled with Ventavis-20 were assessed. In order to compare particle size distribution of the two different solutions, the mass median aerodynamic diameters (MMAD) of the aerosol droplets were determined using laser light scattering (Sympatec™, Clausthal-Zellerfeld, Germany). The measurements (five runs of 1 sec duration, sampling rate 50 ms) were performed without additional air flow, with a distance between mouthpiece and laser beam of 5 cm. The data were analyzed in MIE mode, the density of the nebulized solution was set equal to unit density and thus the measured volume median diameter (VMD) equaled the mass median aerodynamic diameter. The fine particle fraction (FPF) was defined as the mass of particles <5.25 μm in size within the total emitted dose divided by the total emitted dose of aerosol particles. The geometric standard deviation (GSD) was calculated from the laser diffraction values according to the following equation:

$$GSD = \sqrt{\frac{84 \% undersize}{16 \% undersize}}$$

To assess the aerosol volume emitted by one puff from the Respimat™, the drug container of the device was weighed before and after a series of 40 consecutive puffs.

Firstly, the parameters were assessed for the placebo Respimat™. Following the experiments with the placebo Respimat™, the drug container was completely emptied by a syringe. After weighing the drug container, 3.0 ml of Ventavis-20 was filled in the drug container by a syringe. Then the second series of experiments was performed to obtain the aerosol parameters for Respimat™ with Ventavis-20. The results are summarized in the following Table 1:

TABLE 1

| | | | | Volume per |
|---|---|---|---|---|
| | MMAD (μm) | GSD | FPF (%) | puff (μl) |
| Respimat Placebo | 4.28 ± 0.17 | 1.70 ± 0.03 | 66.6 ± 3.6 | 16.6 |
| Respimat Ventavis-20 | 4.21 ± 0.30 | 1.76 ± 0.07 | 66.3 ± 6.0 | 16.9 |

MMAD: mass median aerodynamic diameter,
GSD: geometric standard deviation,
FPF: fine particle fraction,
mean ± standard deviation,
n = 5

### Example 2 Respimat™ and Ilomedin™ 100 μg/ml

In this in vitro nebulization study, placebo Respimat™ was compared to Respimat™ filled with Ilomedin™ 100 μg/ml. The physical aerosol characteristics and the output were assessed as described above (see example 1). Following the experiments with the placebo Respimat™, the drug container was completely emptied by a syringe. After weighing the drug container, 3.0 ml of Ilomedin™ 100 μg/ml was filled in the drug container by a syringe. Then the second series of experiments was performed to obtain the aerosol parameters for Respimat™ filled with Ilomedin™ 100 μg/ml. The results are summarized in the following Table 2:

TABLE 2

| | | | | Volume per |
|---|---|---|---|---|
| | MMAD (μm) | GSD | FPF (%) | puff (μl) |
| Respimat Placebo | 4.09 ± 0.14 | 1.69 ± 0.03 | 69.5 ± 3.1 | 16.3 |
| Respimat Ilomedin-100 | 4.05 ± 0.21 | 1.70 ± 0.04 | 70.3 ± 5.1 | 16.7 |

MMAD: mass median aerodynamic diameter,
GSD: geometric standard deviation,
FPF: fine particle fraction,
mean ± standard deviation,
n = 5

### Example 3 Respimat™ and Ilomedin™ 50 μg/ml

In this in vitro nebulization study, placebo Respimat™ was compared to Respimat™ filled with Ilomedin™ 50 μg/ml. The physical aerosol characteristics and the output were assessed as described above (see example 1). Following the experiments with the placebo Respimat™, the drug container was completely emptied by a syringe. After weighing the drug container, 3.0 ml of Ilomedin™ 50 μg/ml (1.5 ml Ilomedin™ 100 μg/ml diluted by 1.5 ml saline 0.9%) was filled in the drug container by a syringe. Then the second series of experiments was performed to obtain the aerosol parameters for Respimat™ filled with Ilomedin™ 50 μg/ml. The results are summarized in the following Table 3:

TABLE 3

| | | | | Volume per |
|---|---|---|---|---|
| | MMAD (μm) | GSD | FPF (%) | puff (μl) |
| Respimat Placebo | 4.16 ± 0.19 | 1.67 ± 0.08 | 69.2 ± 2.4 | 17.0 |
| Respimat Ilomedin-50 | 4.19 ± 0.14 | 1.68 ± 0.05 | 69.3 ± 3.9 | 16.8 |

MMAD: mass median aerodynamic diameter,
GSD: geometric standard deviation,
FPF: fine particle fraction,
mean ± standard deviation,
n = 5

### Example 4 Medspray™ Wet Aerosol Inhaler and Ventavis-20

In an in vitro nebulization study, the feasibility of delivering iloprost solution by the Medspray™ wet aerosol inhaler was evaluated. The physical aerosol characteristics of Medspray™ wet aerosol inhaler filled either with sodium chloride 0.9% or with Ventavis-20 were assessed. In order to compare particle size distribution of the two different solutions, the mass median aerodynamic diameters (MMAD) of the aerosol droplets were determined using laser light scattering (Sympatec, Clausthal-Zellerfeld, Germany). The measurements (five runs of 1 sec duration, sampling rate 50 ms) were performed without additional air flow, with a distance between mouthpiece and laser beam of 5 cm. The data were analyzed in MIE mode, the density of the nebulized solution was set equal to unit density and thus the measured volume median diameter (VMD) equaled the mass median aerodynamic diameter. The fine particle fraction was defined as the mass of particles <5.25 μm in size within the total emitted dose divided by the total emitted dose of aerosol particles. The geometric standard deviation (GSD) was calculated from the laser diffraction values according to the following equation:

$$GSD = \sqrt{\frac{84 \% undersize}{16 \% undersize}}$$

For the nebulization experiments, 1 ml of physiological saline or Ventavis-20 solution was filled in a 1 ml syringe. A spray nozzle (nozzle diameter 1.7 μm) was then fixed at the tip of the syringe and the aerosol was produced by pressing the solution through the spray nozzle. The results are summarized in the following Table 4:

TABLE 4

| | MMAD (μm) | GSD | FPF (%) |
|---|---|---|---|
| Medspray NaCl 0.9% | 5.04 ± 0.26 | 1.50 ± 0.05 | 56.7 ± 3.6 |
| Medspray Ventavis-20 | 5.15 ± 0.30 | 1.50 ± 0.06 | 55.3 ± 3.8 |

MMAD: mass median aerodynamic diameter,
GSD: geometric standard deviation,
FPF: fine particle fraction,
mean ± standard deviation,
n = 5

### Example 5 Medspray™ Wet Aerosol Inhaler and Ilomedin™ 100 μg/ml

In this in vitro nebulization study, Medspray™ wet aerosol inhalers filled either with sodium chloride 0.9% or with Ilomedin™ 100 μg/ml were compared. The physical aerosol characteristics were assessed as described above (see example 4). The results are summarized in the following Table 5:

TABLE 5

| | MMAD (μm) | GSD | FPF (%) |
|---|---|---|---|
| Medspray NaCl 0.9% | 5.03 ± 0.25 | 1.46 ± 0.05 | 57.3 ± 2.7 |
| Medspray Ilomedin-100 | 4.98 ± 0.35 | 1.49 ± 0.04 | 60.8 ± 3.4 |

MMAD: mass median aerodynamic diameter,
GSD: geometric standard deviation,
FPF: fine particle fraction,
mean ± standard deviation,
n = 5

### Example 6 Medspray™ Wet Aerosol Inhaler and Ilomedin™ 50 μg/ml

In this in vitro nebulization study, Medspray™ wet aerosol inhalers were filled either with 1 ml sodium chloride 0.9% or with 1 ml Ilomedin™ 50 μg/ml (0.5 ml Ilomedin™ 100 μg/ml diluted by 0.5 ml NaCl 0.9%). The physical aerosol characteristics were assessed as described above (see example 4). The results are summarized in the following Table 6:

TABLE 6

| | MMAD (μm) | GSD | FPF (%) |
|---|---|---|---|
| Medspray NaCl 0.9% | 5.16 ± 0.29 | 1.54 ± 0.05 | 54.9 ± 5.0 |
| Medspray Ilomedin-50 | 5.25 ± 0.36 | 1.54 ± 0.06 | 50.5 ± 4.4 |

MMAD: mass median aerodynamic diameter,
GSD: geometric standard deviation,
FPF: fine particle fraction,
mean ± standard deviation,
n = 5

### Example 7 Medspray™ Wet Aerosol Inhaler with Smaller Nozzle Diameters (e.g. 1.5 or 1 μm)

By changing the diameter of the holes in the spray nozzle, the resulting droplet size distribution can be adjusted to the specific requirement of regional drug deposition within the respiratory tract. Pressing aqueous solution through the nozzle results in a jet which automatically breaks up in droplets (Rayleigh breakup), with droplet sizes theoretically twice the size of the hole. When using nozzle diameters of 1.5 or 1 μm, the size range of the droplets is within 2 μm to 5.0 μm. In addition, deep lung aerosol deposition is enhanced by the low velocity of the aerosol. Peripheral deposition can further be increased by implementing a flow limit for inhalation (e.g. by valves) ensuring slow air flow during inhalation.

The examples demonstrate that by use of the soft mist inhalers Respimat™ or Medspray™ wet aerosol inhaler an iloprost-containing aerosol suitable for deep lung deposition can be provided.

### Dosing

Provided herein are doses of 0.4 μm to 5 μg of iloprost delivered at the mouthpiece of the soft mist inhalers Respimat™ or Medspray™ wet aerosol inhaler within one to ten puffs for a single PRN inhalation treatment. Also preferred are single doses of about 2.5 μg and about 5 μg, as currently used in the therapy of pulmonary arterial hypertension.

The following is a non-exhaustive list of possible combinations of iloprost drug concentration and emitted volume per puff suitable to deliver the claimed dose for one PRN treatment session.

### Example 8 Respimat™ and Iloprost 100 μg/ml (Respimat 100)

Table 7 depicts the delivered iloprost dose at the mouthpiece of the soft mist inhaler Respimat™ depending on the emitted aerosol volume and number of puffs when using an iloprost drug concentration of 100 μg/ml.

TABLE 7

| | Volume | Number of | Iloprost dose at the |
|---|---|---|---|
| | (μl) | puffs | mouthpiece (μg) |
| Respimat 100 | 10 | 1, 2, 3, 4, 5 | 1, 2, 3, 4, 5 |
| Respimat 100 | 15 | 1, 2, 3 | 1.5, 3, 4.5 |
| Respimat 100 | 20 | 1, 2 | 2, 4 |
| Respimat 100 | 22.5 | 1, 2 | 2.25, 4.5 |
| Respimat 100 | 25 | 1, 2 | 2.5, 5 |

### Example 9 Respimat™ and Iloprost 20 μg/ml (Respimat 20)

Table 8 depicts the delivered iloprost dose at the mouthpiece of the soft mist inhaler Respimat™ depending on the emitted aerosol volume and number of puffs when using an iloprost drug concentration of 20 μg/ml.

TABLE 8

| | Volume | Number of | Iloprost dose at the |
|---|---|---|---|
| | (μl) | puffs | mouthpiece (μg) |
| Respimat 20 | 20 | 3, 6, 9 | 1.2, 2.4, 3.6 |
| Respimat 20 | 25 | 2, 4, 6, 8, 10 | 1, 2, 3, 4, 5 |

It is within the scope of the present invention to use iloprost drug concentrations in the range from 20 μg/ml to 100 μg/ml. In another preferred embodiment iloprost drug concentration is 50 μg/ml, resulting in a delivered dose of 1 μg in 1 puff, 2 μg in 2 puffs, 3 μg in 3 puffs, 4 μg in 4 puffs and 5 μg in 5 puffs, when the emitted volume per puff of the Respimat™ is set to 20 μl.

### Example 10 Medspray™ Wet Aerosol Inhaler and Iloprost 100 or 50 μg/ml (Medspray 100 or 50)

Table 9 depicts the delivered iloprost dose at the mouthpiece of the soft mist inhaler Medspray™ depending on the emitted aerosol volume and number of puffs when using an iloprost drug concentration of 100 μg/ml or 50 μg/ml.

TABLE 9

| | Volume | Number of | Iloprost dose at the |
|---|---|---|---|
| | (μl) | puffs | mouthpiece (μg) |
| Medspray 100 | 20 | 1, 2 | 2, 4 |
| | 22.5 | 1, 2 | 2.25, 4.5 |
| | 25 | 1, 2 | 2.5, 5 |
| Medspray 50 | 20 | 1, 2, 3, 4, 5 | 1, 2, 3, 4, 5 |
| | 25 | 1, 2, 3, 4 | 1.25, 2.5, 3.75, 5 |
| | 50 | 1, 2 | 2.5, 5 |

### Example 11 Medspray™ Wet Aerosol Inhaler and Iloprost 20 μg/ml (Medspray 20)

Table 10 depicts the delivered iloprost dose at the mouthpiece of the soft mist inhaler Medspray™ depending on the emitted aerosol volume and number of puffs when using an iloprost drug concentration of 20 μg/ml.

TABLE 10

| | Volume | Number of | Iloprost dose at the |
|---|---|---|---|
| | (μl) | puffs | mouthpiece (μg) |
| Medspray 20 | 50 | 1, 2, 3, 4, 5 | 1, 2, 3, 4, 5 |
| | 40 | 1, 2, 3, 4, 5 | 0.8, 1.6, 2.4, 3.2, 4.0 |

It is within the scope of the present invention to use iloprost drug concentrations in the range from 20 μg/ml to 100 μg/ml.

The drug containers of the Respimat™ and the Medspray™ wet aerosol inhaler can be filled with 0.5 to 5 ml of the claimed iloprost solutions. Preferentially, the filling volume is limited to a range of 0.5 to 2 ml, or 0.5 to 1 ml in order to avoid overdosage. The drug containers may contain 0.2 to 11 times the daily maximum inhaled iloprost dose of 45 μg (according to the Ventavis™ product information), preferentially 1 to 5 times, or 2 to 4 times. In one embodiment, the soft mist inhalers Respimat™ or Medspray™ are disposable, i.e. the soft mist inhalers are discarded as a whole after delivery of a preset number of puffs. In another embodiment, only the empty drug containers are replaced, with the devices being reused several times (e.g. three to five times) before being replaced.

Patients treated with the compositions and methods disclosed herein suffer from pulmonary hypertension or from other disorders of the pulmonary vasculature or pulmonary circulation. For example, the subjects may belong to one of the following five groups of pulmonary hypertension according to the WHO: Group 1, Pulmonary arterial hypertension (PAH) including subclasses 1.1 Idiopathic PAH, 1.2 Heritable PAH, 1.3 Drug- and toxin-induced PAH, 1.4 PAH associated with 1.4.1 Connective tissue disease, 1.4.2 HIV infection, 1.4.3 Portal hypertension, 1.4.4 Congenital heart disease, 1.4.5 Schistosomiasis, 1.5 PAH long-term responders to calcium channel blockers; 1.6 PAH with overt features of venous/capillaries (PVOD/PCH) involvement and 1.7 Persistent PH of the newborn syndrome; Group 2, Pulmonary hypertension due to left heart disease including subclasses 2.1 PH due to heart failure with preserved LVEF, 2.2 PH due to heart failure with reduced LVEF, 2.3 Valvular heart disease and 2.4 Congenital/acquired cardiovascular conditions leading to post-capillary PH; Group 3, Pulmonary hypertension due to lung disease and/or hypoxia including subclasses 3.1 Obstructive lung disease 3.2 Restrictive lung disease, 3.3 Other lung disease with mixed restrictive/obstructive pattern, 3.4 Hypoxia without lung disease and 3.5 Developmental lung disorders; Group 4, Pulmonary hypertension due to pulmonary artery obstructions including subclasses 4.1 Chronic thromboembolic PH and 4.2 Other pulmonary artery obstructions; Group 5, Pulmonary hypertension with unclear and/or multifactorial mechanisms including subclasses 5.1 Hematological disorders 5.2 Systemic and metabolic disorders, 5.3 Others and 5.4 Complex congenital heart disease disorder. Preferably, the subject belongs to Group 1 or Group 4 PH to benefit from the provided methods and compositions for administering inhalable iloprost as PRN therapy. The subject may belong to functional class I, class II, class Ill or class IV according to the functional classification of pulmonary hypertension of the World Health Organization, modified after the New York Heart Association functional classification.

The subject may have no medication, or receive supportive therapy such as oral anticoagulants, diuretics, oxygen, digoxin. In addition, therapy may include high-dose calcium channel blockers or specific drugs approved for PH, encompassing endothelin receptor antagonists such as ambrisentan (oral), bosentan (oral) or macitentan (oral), phosphodiesterase type 5 inhibitors and guanylate cyclase stimulators or activators such as sildenafil (oral, intravenous), tadalafil (oral), vardenafil (oral) or riociguat (oral), prostacyclin analogues and prostacyclin receptor agonists such as beraprost (oral), epoprostenol (intravenous), iloprost (aerosol, intravenous), treprostinil (aerosol, subcutaneous, intravenous, oral) or selexipag (oral). These mainly vasodilatory drugs may be administered to the subject as monotherapy or as combination therapy using two or more drugs simultaneously. Also included in the present invention is the use of future PH-specific drugs as background therapy, with such drugs mainly focusing on typical characteristics of pulmonary vascular remodeling.

The PRN iloprost inhalation disclosed herein may be administered to therapy-naïve patients or to patients on supportive therapy. In addition, PRN iloprost therapy may be on top of chronic background therapy using one or more PH-specific drugs.

The pharmacodynamic profile of inhaled iloprost is well-known. When administered by conventional nebulizers over a time period of 10 minutes, the maximum therapeutic effects on hemodynamic parameters of pulmonary circulation are observed approximately 5 minutes after end of inhalation. Surprisingly, significant vasodilatory effects in the pulmonary vasculature are already seen within only one minute after rapid iloprost inhalation by 2 or 4 puffs à 1.25 μg iloprost, maintaining pulmonary selectivity of this approach. The observed pharmacodynamic profile according example 12 qualifies inhaled iloprost as ideal candidate for PRN therapy of pulmonary hypertension.

### Example 12 Iloprost PRN in Patients with Pulmonary Hypertension

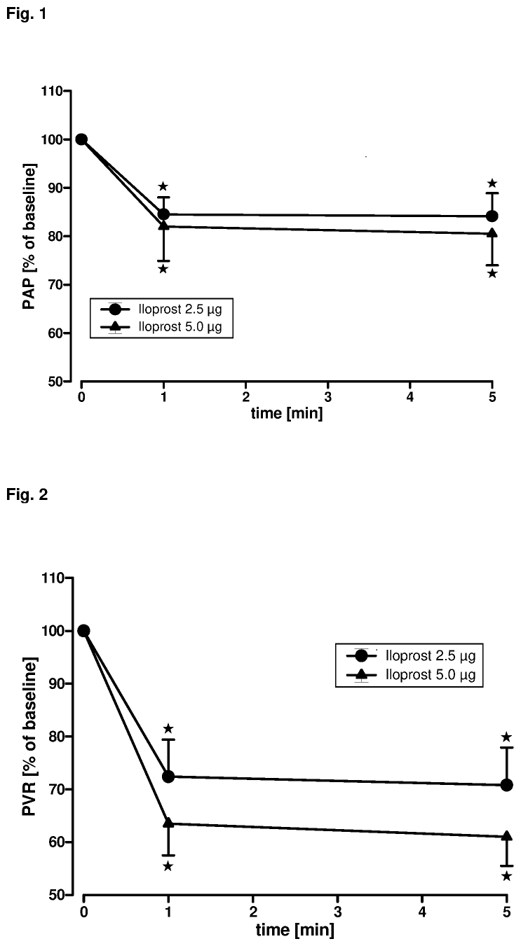

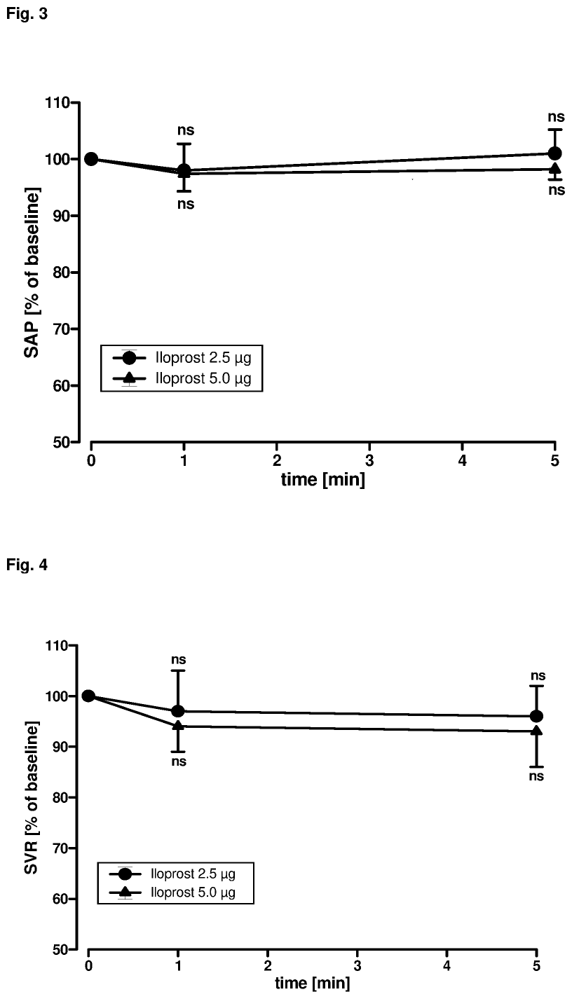

A clinical pilot study to evaluate the feasibility of iloprost PRN was conducted with 4 patients. Eligible were male and female patients with pulmonary arterial hypertension in the age from 18 to 70 years having a mean pulmonary arterial blood pressure (PAP) higher than 25 mm Hg, a pulmonary vascular resistance (PVR) higher than 240 dyn*s*cm−5, a central venous pressure (CVP) higher than 3 mm Hg, and a pulmonary capillary wedge pressure (PCWP) lower than 12 mm Hg. Patients were therapy-naïve, or were receiving PH-specific medication (endothelin receptor antagonists, phosphodiesterase type 5 inhibitors, prostacyclin analogues) alone or in combination. Patients were monitored by ECG, pulse oxymetry, and non-invasive blood pressure measurement. An intracardiac catheter was introduced into the distal pulmonary artery to measure PAP, CVP, PCWP, and cardiac output. Also measured were the heart rate, systemic arterial pressure (SAP), systemic vascular resistance (SVR), central arterial and venous blood gases. After the initial determination of all parameters the reactivity of the pulmonary vasculature to oxygen (2 to 4 L/min) and nitric oxide (20 ppm) was tested. Subsequently, patients inhaled a single dose of 2.5 μg of nebulized iloprost in two breaths, using a prototype of a soft mist inhaler filled with iloprost in a concentration of 50 μg/ml. Hemodynamic parameters and clinical condition of the patients were assessed before and 1, 5, 15 and 30 minutes after inhalation. In the absence of adverse effects, a second inhalation maneuver with 4 breaths corresponding to a dose 5 μg iloprost was performed, again followed by an observation period of at least 30 minutes. In result, it was found that all patients tolerated the treatment well. The therapeutic pulmonary vascular effects (as indicated by PAP and PVR changes, see FIG. 1 and FIG. 2) were already registered one minute after finishing the inhalation maneuver, without significant systemic side effects (as indicated by SAP and SVR changes, see FIG. 3 and FIG. 4).

PRN iloprost is intended for the acute treatment of PH on an as-needed basis, e.g. to facilitate improvements in exercise tolerance and activities of daily living, to reduce symptoms of the disease, or to overcome acute pulmonary hypertensive crises. By means of the portable soft mist inhalers Respimat™ or Medspray™ patients can inhale at anytime and anywhere an effective dose of PRN iloprost (up to 5 μg) up to a maximum of nine times per day, resulting in a maximum daily iloprost dose of 45 μg. In the provided methods, when a patient is envisaging physical exertion or strenuous activities, or when a patient is perceiving dyspnea, fatigue, dizziness, chest pressure or pain, edema formation, cyanosis, tachycardia or heart palpitations, the subject administers via inhalation PRN iloprost preferably 0 to 15 minutes before initiating such activity, or during such activity or episode. Patients can inhale one puff or several puffs closely spaced, or several puffs within an interval of 15 seconds to 5 minutes, according to individual need, desired effects and tolerability. Patients not receiving prostanoids on a daily regular basis as their PH-specific therapy will usually inhale 0.4 to 2.5 μg total iloprost dose per PRN treatment cycle, whereas patients chronically treated by prostanoids, especially inhaled prostanoids, will inhale 0.4 to 5 μg per PRN treatment cycle, preferentially 2.5 to 5 μg. The methods provided herein offer to PH patients the possibility to cope with the requirements and challenges of daily life activities and to improve quality of life by self-administration of an effective dose of inhaled iloprost as needed.

1. A method of treating a patient suffering from pulmonary hypertension, comprising:
(a) providing a portable and pre-filled soft mist inhaler adapted for delivering an effective amount of Iloprost; and
(b) administering to the patient the effective amount of Iloprost by inhalation on an as-needed basis.

2. A method of claim 1, wherein the effective amount of inhaled Iloprost is 0.4 μg to 5 μg per pro re nata treatment cycle, delivered in one to ten single puffs from the soft mist inhaler.

3. A method of claim 1, wherein the onset of action of inhaled Iloprost in the pulmonary circulation is within one minute after completing the PRN inhalation, with maintained pulmonary selectivity preventing significant side effects in the systemic circulation.

4. A method of claim 1, wherein the soft mist inhaler is the Respimat™.

5. A method of claim 1, wherein the soft mist inhaler Respimat™ is filled with Iloprost solution containing Iloprost in concentrations of 20 μg/ml to 100 μg/ml, additionally containing trometamol, ethanol 96%, sodium chloride, hydrochloric acid (for pH adjustment) and water for injections.

6. A method of claim 1, wherein the emitted aerosol volume is 10 μl to 25 μl, facilitating one to ten puffs to deliver 0.4 μg to 5 μg of inhaled Iloprost using the Respimat™.

7. A method of claim 1, wherein the soft mist inhaler is the Medspray™ wet aerosol inhaler.

8. A method of claim 1, wherein the soft mist Medspray™ wet aerosol inhaler is filled with Iloprost solution containing Iloprost in concentrations of 20 g/ml to 100 μg/ml, additionally containing trometamol, ethanol 96%, sodium chloride, hydrochloric acid (for pH adjustment) and water for injections.

9. A method of claim 1, wherein the emitted aerosol volume is 20 μl to 50 μl, facilitating one to five puffs to deliver 0.4 μg to 5 μg of inhaled Iloprost using Medspray™ wet aerosol inhaler.

10. A method of claim 4, wherein the volume of the drug container of the Respimat™ or Medspray™ wet aerosol inhaler is 0.5 ml to 5, preferably of 0.5 to 2 ml, or 0.5 to 1 ml. The drug containers therefore contain 0.2 to 11 times the daily maximum approved inhaled Iloprost dose of 45 μg, preferentially 2 to 5 times, or 2 to 4 times.

11. A method of claim 1, wherein the produced aerosol from the soft mist inhalers is suitable for peripheral lung deposition, i.e. the volume median diameter (VMD) or mass median aerodynamic diameter (MMAD) of the aerosol is in a range of 1 to 6 μm, preferably 2 to 5.5 μm; and the geometric standard deviation (GSD) is in a range of 1.2 to 2, preferably 1.2 to 1.8.

12. A method of claim 1, wherein the soft mist inhalers Respimat™ or Medspray™ are disposable.

13. A method of claim 1, wherein the empty drug containers are replaced, with the soft mist inhalers Respimat™ or Medspray™ being reused several times (preferably three to five times) before also being replaced.

14. A method of claim 1, wherein the patients suffering from pulmonary hypertension is
(a) therapy-naïve,
(b) on supportive therapy and/or
(c) receiving chronic therapy with one or more approved PH-specific drugs selected from the group of endothelin receptor antagonists (e.g. ambrisentan, bosentan, macitentan), of phosphodiesterase type 5 inhibitors and guanylate cyclase stimulators (e.g. sildenafil, tadalafil, vardenafil, or riociguat), of prostacyclin analogues and prostacyclin receptor agonists (e.g. beraprost, epoprostenol, iloprost, treprostinil, or selexipag), and/or
(d) receiving disease-modifying drugs addressing vascular remodeling, alone or in combination with therapy according to (b) or (c).

15. A method of claim 1, wherein the effective amount of inhaled Iloprost is 0.4 μg to 5 μg per pro re nata treatment cycle, delivered in one to four single puffs.
